(19) United States
(12) Patent Application Publication
(10) Pub. No.: US 2026/0335210 A1
(43) Pub. Date: Oct. 29, 2026

(54) LIQUID CLEANSING COMPOSITION

(71) Applicant: Conopco, Inc., d/b/a UNILEVER, Englewood Cliffs, NJ (US)

(72) Inventors: Marie Lynn McCOLGAN, Norwalk, CT (US); Leonora YOKUBINAS, Stratford, CT (US)

(73) Assignee: Conopco, Inc., d/b/a UNILEVER, Englewood Cliffs, NJ (US)

(21) Appl. No.: 19/099,068

(22) PCT Filed: Jul. 12, 2023
(86) PCT No.: PCT/EP2023/069379

§ 371 Date: Jan. 28, 2025

Related U.S. Application Data
(60) Provisional application No. 63/394,916, filed on Aug. 3, 2022.

(30) Foreign Application Priority Data
Sep. 21, 2022 (EP) 22196949.6

(51) Int. Cl. A61K 8/46; A61K 8/34; A61K 8/365; A61K 8/44; A61Q 5/02; A61Q 19/10

(52) U.S. Cl. CPC A61K 8/466; A61K 8/365; A61K 8/442; A61Q 5/02; A61Q 19/10; A61K 8/345; A61K 2800/34; A61K 2800/596; A61K 2800/805

(57) ABSTRACT

The present invention relates to liquid cleansing compositions suitable for topical application for cleansing the human body, especially the skin and hair. In particular, the invention relates to compositions which are preferably sulfate free, thickening agent free, and comprise an organic acid. Surprisingly, the compositions lather appreciably, are stable and are very mild.

### FIELD OF THE INVENTION

The present invention relates to liquid cleansing compositions suitable for topical application for cleansing the human body, especially the skin and hair. In particular, the invention relates to compositions which are preferably sulfate free, thickening agent free, and comprise an organic acid. Surprisingly, the compositions lather appreciably, are stable and are very mild.

### BACKGROUND OF THE INVENTION

Consumers seek personal cleansing compositions that are mild and able to deliver a creamy lather so that skin and hair feel soft and smooth after a single wash. In addition to soft and smooth skin and hair, such consumers desire personal cleansing compositions comprising an exfoliator to aid in smoothing the skin and removing dead skin cells of the stratum corneum. Popular cleansing compositions with exfoliators, particularly chemical exfoliants, such as organic acids like alpha hydroxy acids, can pose challenges to stability of the overall composition, for example, by precipitating out and negatively impacting formulation viscosity and lather characteristics. Efforts to remedy such issues typically include the use of synthetic thickening agents whereby the addition of such ingredients resolves the viscosity issues but often impacts the sensory benefits delivered to the consumer after use. It is thus difficult to formulate exfoliating personal cleansing compositions with organic acids which are stable and mild with consumer desirable lather and viscosity characteristics, especially ones substantially free of thickening agents.

In the disclosed cleansing composition, it has been found that for personal cleansing compositions comprising certain anionic and zwitterionic surfactants, and organic acid, the cleansing compositions, surprisingly, are stable, have a desirable viscosity, and deliver consumer desirable sensory characteristics when formulated substantially free of thickening agent, electrolytes, or both. The cleansing composition disclosed herein is also environmentally sustainable since synthetic thickeners that are often not biodegradable may be avoided.

### SUMMARY OF THE INVENTION

Accordingly, in a first aspect, disclosed herein is a cleansing composition comprising anionic surfactant, zwitterionic surfactant, and organic acid and/or salt or ester thereof, wherein the composition has a pH ranging from 4.0 to 6.0, or 4.15 to 4.75, or 4.25 to 5.5, and wherein the composition is substantially free of thickening agent, electrolytes, or both.

In a second aspect of the composition disclosed herein, there is provided a use of a composition comprising anionic surfactant, zwitterionic surfactant, and organic acid and/or salt or ester thereof for formulating a thickened and mild cleansing composition for smoothing skin wherein the composition is substantially free of thickening agent, electrolytes, or both.

In a third aspect, disclosed is a method of cleansing for gentle exfoliation of the skin comprising the steps of:
(a) contacting the skin with a cleansing composition comprising anionic surfactant, zwitterionic surfactant, and organic acid and/or salt or ester thereof, wherein the composition is substantially free of thickening agent, electrolytes, or both; and
(b) removing, or washing or wiping, the composition off the skin.

All other aspects of the present cleansing compositions will become more readily apparent upon considering the detailed description and examples which follow.

### DETAILED DESCRIPTION OF THE INVENTION

Unless explicitly stated otherwise, all ranges described herein are meant to include all ranges subsumed therein. Except in the operating and comparative examples, or where otherwise explicitly indicated, all numbers used in this description indicating amounts, or ratios of materials and/or use thereof are to be understood as modified by the word “about.”

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above. The term comprises is also meant to encompass the terms consisting essentially of and consisting of. For the avoidance of doubt, a cleansing composition comprising an anionic surfactant, a zwitterionic surfactant, and an organic acid is meant to include a composition consisting essentially of the same and a composition consisting of the same. As to the percentages used herein, the same are meant to be by weight of ingredient active, unless noted otherwise.

Esters of organic acids includes, for example, those derived from a carboxylic acid and a C₂ to C₈ alkanol, linear, branched, and/or functionalized with a hydroxy group. Salts of acids include those having a sodium (Na⁺), potassium (K⁺), or magnesium (Mg⁺) counter ion or other solubilizing cation as defined herein.

The disclosure, as found herein, is to be considered to cover all embodiments as found in the claims as being multiply dependent upon each other irrespective of the fact that claims may be found without multiple dependency or redundancy.

Cleansing compositions disclosed herein comprise at least one anionic surfactant. Desirable anionic surfactants are non-soap.

Taurates may be used as an anionic surfactant, the same is limited only to the extent that it is one suitable for use in a consumer product. Illustrative examples of the taurate surfactant that may be used in the disclosed cleansing composition include, for example, those which are acylamides of taurine or N-methyltaurine, and salts thereof. For example, taurates suitable for use are acyl taurates represented by the general formulae:

RC(O)N(R¹)(CH₂)_{y}SO₃M (I), and

RC(O)N(R¹)CH₂CH₂SO₃M (II),

where R is C₆ to C₃₀, more particularly, C₆ to C₂₄ alkyl, y is 2 or 3, R¹ is hydrogen or methyl, and M is hydrogen or a solubilizing cation such as, for example, hydrogen, ammonium, alkali metal cation, a lower C₁ to C₄, alkanol ammonium cation and/or a basic amino acid cation. In one embodiment, R is C₈ to C₁₈ alkyl. In another embodiment at least half of the R groups are C₈-C₁₈ alkyl. In still another embodiment at least half of the R groups are C₁₀ to C₁₄ alkyl. R may be saturated or unsaturated. In yet another embodiment R¹ is methyl.

Illustrative acyl taurates that may be used as an anionic surfactant of the disclosed cleansing composition include, for example, taurates commonly known as sodium methyl lauroyl taurate, sodium methyl myristoyl taurate, sodium methyl cocoyl taurate, sodium methyl oleoyl taurate, sodium cocoyl taurate, mixtures thereof or the like. In an embodiment, the taurate used is sodium methyl lauroyl taurate. Anionic surfactants like taurates may be present in an amount of 0.001 to 20%, or 0.01 to 15%, or 0.5 to 12%, 1 to 10%, or 2 to 6% by weight of the cleansing composition. Taurate may comprise from 50 to 90% by weight of the total anionic surfactant. In another aspect, taurate makes up 100% by weight of the total anionic surfactant.

Other anionic surfactants may be used in the disclosed cleansing composition in addition to taurate. These additional anionic surfactants may include alkyl sulfosuccinates (including mono- and dialkyl, e.g., C₆-C₂₂ sulfosuccinates); alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, acyl glutamates, glycinates, alaninates, and the like.

Sulfosuccinates may be monoalkyl sulfosuccinates having the formula:

R²OC(O)CH₂CH(SO₃M)CO₂M;

and amide-MEA sulfosuccinates of the formula:

R²CONHCH₂CH₂OC(O)CH₂CH(SO₃M)CO₂M

wherein R² ranges from C₈-C₂₂ alkyl.

Sarcosinates are generally indicated by the formula:

R³CON(CH₃)CH₂CO₂M,

wherein R³ ranges from C₈-C₂₀ alkyl.

Glycinates generally have the formula:

R⁴CONR⁵CH₂CO₂M,

wherein R⁴ is a C₈-C₂₄ alkyl, R⁵ is hydrogen or CH₃.

Acyl glutamates generally have the formula:

R⁶CONH(CHCO₂H)CH₂CH₂CO₂M or

R⁶CONH(CHCO₂M)CH₂CH₂CO₂H,

wherein R⁶ is a C₈-C₂₀ alkyl or alkenyl.

M is a solubilizing cation as previously described.

In an aspect of the cleansing composition, other desirable anionic surfactants include 2-acrylamido-2-methylpropane sulfonic acid, ammonium lauryl sulfate, ammonium perfluorononanoate, potassium lauryl sulfate, sodium alkyl sulfate, sodium dodecyl sulfate, sodium laurate, sodium laureth sulfate, sodium lauroyl sarcosinate, sodium stearate, sodium sulfosuccinate esters, sodium lauroyl glutamate, sodium myristoyl glutamate, sodium stearoyl glutamate, sodium cocoyl glutamate, potassium myristoyl glutamate, disodium cocoyl glutamate, sodium cocoyl glycinate, potassium cocoyl glycinate, disodium 2-sulfolaurate, sodium stearoyl lactylate, sodium cocoyl alaninate, or a combination thereof. Such anionic surfactants are commercially available from suppliers like Galaxy Surfactants, Clariant, Sino Lion, Stepan Company, and Innospec. In another aspect of the cleansing composition, the anionic surfactant used is an isethionate, a taurate, a sodium alkyl sulfate like sodium lauryl sulfate, an acyl glutamate, a glycinate, a lactylate, an alaninate, or a mixture thereof. In another aspect of the cleansing composition, the anionic surfactants for use include sodium cocoyl alaninate, sodium cocoyl isethionate, sodium lauryl isethionate, sodium methyl cocoyl taurate, sodium methyl lauroyl taurate, sodium lauryl sulfate, sodium lauroyl glutamate, sodium cocoyl glutamate, sodium cocoyl glycinate, potassium cocoyl glycinate, disodium lauryl sulfosuccinate, sodium stearoyl lactylate, or a combination thereof. In one embodiment, the cleansing composition is substantially free of sulfate-based surfactants (i.e., is sulfate free). In another embodiment, the cleansing composition is free of sulfate-based surfactants. “Substantially free of,” as used herein, is intended to mean comprising less than 5% by weight, or less than 2.5% by weight, or less than 1% by weight, or less than 0.5% by weight, or less than 0.1% by weight, or 0% by weight of the ingredient.

As to the zwitterionic surfactants employed in the present cleansing composition, such surfactants include at least one acid group. Such an acid group may be a carboxylic or a sulphonic acid group. They often include quaternary nitrogen, and therefore, can be quaternary amino acids. They should generally include an alkyl or alkenyl group of 7 to 18 carbon atoms and generally comply with an overall structural formula:

R⁷—[—C(O)—NH(CH₂)_{q}—]ᵣ—N⁺(R⁸)(R⁹)-A-B,

where R⁷ is alkyl or alkenyl of 7 to 18 carbon atoms; R⁸ and R⁹ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Desirable zwitterionic surfactants for use in the cleansing composition disclosed herein and within the above general formula include simple betaines of formula:

R⁷—N⁺(R⁸)(R⁹)—CH₂CO₂

and amido betaines of formula:

R⁷—CONH(CH₂)ₜ—N⁺(R⁸)(R⁹)—CH₂CO₂—,

where t is 2 or 3.

In both formulae R⁷, R⁸ and R⁹ are as defined previously. R⁷ may, in particular, be a mixture of C₁₂ and C₁₄ alkyl groups derived from coconut oil so that at least half, or at least three quarters, of the groups R⁷ have 10 to 14 carbon atoms. R⁸ and R⁹ are preferably methyl.

A further possibility is that the zwitterionic surfactant is a sulphobetaine of formula:

R⁷—N⁺(R⁸)(R⁹)—(CH₂)₃SO₃⁻ or

R⁷—CONH(CH₂)ᵤ—N⁺(R⁸)(R₉)—(CH₂)₃SO₃⁻,

where u is 2 or 3, or variants of these in which —(CH₂)₃SO₃⁻ is replaced by —CH₂C(OH)(H)CH₂SO₃⁻.

In these formulae, R⁷, R⁸ and R⁹ are as previously defined.

Illustrative examples of the zwitterionic surfactants desirable for use include betaines such as lauryl betaine, betaine citrate, cocodimethyl carboxymethyl betaine, cocamidopropyl betaine, coco alkyldimethyl betaine, and laurylamidopropyl betaine. An additional zwitterionic surfactant desirable for use includes cocamidopropyl sultaine, for example, cocamidopropyl hydroxysultaine. Preferred zwitterionic surfactants include lauryl betaine, betaine citrate, sodium hydroxymethylglycinate, (carboxymethyl)dimethyl-3-[(1-oxododecyl)amino]propylammonium hydroxide, coco alkyldimethyl betaine, (carboxymethyl)dimethyloleylammonium hydroxide, cocamidopropyl betaine, (carboxymethyl)dimethyloleylammonium hydroxide, cocamidopropyl betaine, (carboxylatomethyl)dimethyl(octadecyl)ammonium, cocamidopropyl hydroxysultaine, or a combination thereof. Such surfactants are made commercially available from suppliers like Stepan Company, Solvay, Evonik and the like and it is within the scope of the cleansing compositions disclosed herein to employ mixtures of the aforementioned surfactants. In one particular embodiment, the zwitterionic surfactant comprises cocamidopropyl betaine. The cleansing composition comprises to 5 to 40%, or 10 to 35%, or 12 to 30%, or 15 to 25% zwitterionic surfactant by weight of the cleansing composition. In one aspect, the zwitterionic surfactant may comprise from 50 to 90% cocamidopropyl betaine by weight of the zwitterionic surfactant in the total surfactant system. In another aspect, cocamidopropyl betaine makes up 100% by weight of the zwitterionic surfactant in the total surfactant system.

Nonionic surfactants may optionally be used in the cleansing composition. When used, nonionic surfactants are typically used at levels as low as 0.01, 0.1, 1 or 2% by weight and at levels as high as 6, 8, 10 or 12% by weight. The nonionic surfactants which may be used include, in particular, the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

In an aspect, nonionic surfactants can include fatty acid/alcohol ethoxylates having the following structures a) HOCH₂(CH₂)ₛ(CH₂CH₂O)_{c}H or b) HOOC(CH₂)ᵥ(CH₂CH₂O)_{d}H; where s and v are each independently an integer up to 18; and c and d are each independently an integer from 1 or greater. In an aspect, s and v can be each independently 6 to 18; and c and d can be each independently 1 to 30. Other options for nonionic surfactants include those having the formula HOOC(CH₂)ᵢ—CH═CH—(CH₂)ₖ(CH₂CH₂O)_{z}H, where i, k are each independently 5 to 15; and z is 5 to 50. In another aspect, i and k are each independently 6 to 12; and z is 15 to 35.

The nonionic surfactant may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Illustrative examples of nonionic surfactants that can optionally be used in the cleansing compositions disclosed herein include, but are not limited to, polyglycoside, cetyl alcohol, cocamide monoethanolamine (MEA), decyl glucoside, lauryl glucoside, octaethylene glycol monododecyl ether, n-octyl beta-d-thioglucopyranoside, octyl glucoside, oleyl alcohol, polysorbate, sorbitan, stearyl alcohol, or a combination thereof.

Among the organic acids desirable to include in cleansing compositions disclosed herein, any organic acid commonly employed in personal care or pharmaceutical compositions may be selected. In one embodiment, the organic acid is a C₂-C₂₂ carboxylic acid, or C₂-C₁₈ carboxylic acid, or C₂-C₁₆ carboxylic acid. It is desirable that the organic acid is soluble in water. Also desirable are any acids having an acid dissociation constant (pKₐ) from 2 to 12, or 2 to 8, or 2 to 6. Desirable organic acids for use in the disclosed cleansing compositions may comprise, in a nonlimiting manner, independently or in combinations thereof, acetic acid, adipic acid, ascorbic acid, aspartic acid, benzoic acid, cinnamic acid, citric acid, fumaric acid, glutamic acid, glutaric acid, glycolic acid, hydroxybenzoic acid, hydroxycinnamic acid, lactic acid, maleic acid, malic acid, malonic acid, mandelic acid, nicotinic acid, phenylacetic acid, pimelic acid, salicylic acid, sorbic acid, tartaric acid, tartronic acid, tropic acid, gluconic acid, lactobionic acid, and organic acid and/or salt or ester thereof. Organic acid salts, as used herein, includes carboxylate salts of an organic acid (i.e., sodium carboxylates). Organic acid derivatives, as used herein, includes esters (i.e., carboxylate esters) derived from an organic acid and a C₂-C₈ alkanol, or C₂-C₆ alkanol, in particular, C₂-C₆ alkyl esters. In an aspect of the disclosed cleansing composition, the C₂-C₆ alkyl group of such esters may have additional substituents such as one or more hydroxy groups. In another aspect of the disclosed cleansing composition, the organic acid comprises ascorbic acid, citric acid, glycolic acid, lactic acid, malic acid, mandelic acid, tartaric acid, and/or salts or ester thereof. In yet another aspect, the organic acid comprises citric acid, glycolic acid, lactic acid, mandelic acid, and/or salt or ester thereof. In still another aspect, the organic acid is an alpha hydroxy acid, beta hydroxy acid, polyhydroxy acid, or mixtures thereof. In yet another aspect, the organic acid is an alpha hydroxy acid. The cleansing composition comprises 0.001 to 10%, or 0.01 to 8%, or 0.1 to 7%, or 0.5 to 6%, or 1 to 6% active organic acid and/or salts or ester thereof by weight of the cleansing composition.

Now, it has unexpectedly been determined that the cleansing compositions disclosed herein are stable and have a desirable viscosity when substantially free of one or more thickening agents. Such thickening agents include polysaccharides, which comprise of starches, natural/synthetic gums and cellulosics. Illustrative but nonlimiting starches include tapioca starch, cornstarch, potato starch, aluminum starch octenylsuccinate, and sodium hydroxypropyl starch phosphate. Illustrative but nonlimiting gums include acacia senegal gum, agar, agarose, biosaccharide gum, carrageenan, cellulose, gellan gum, guar gum, hyaluronic acid, pectin, sclerotium, xanthan, and combinations thereof. Illustrative but nonlimiting cellulosics include hydroxypropyl cellulose, hydroxypropyl methylcellulose, ethylcellulose, and sodium carboxy methylcellulose. Such thickening agents may also include citric fiber and synthetic polymers functioning as thickening agents, including Carbomers, polyethylene glycol distearates, polyacrylates, polyacrylamides, polymethacrylate, acrylate copolymer, methacrylate copolymer, acrylamide copolymer, taurate copolymers, acryloyldimethyltaurate copolymer, and mixtures thereof. Thickening agents, as used herein, may also include cationic polysaccharide polymers, specifically cationic guar gum derivatives, such as, but are not limited to, guar hydroxypropyltrimonium chloride. The compositions are unexpectedly stable in the absence of the above.

The cleansing compositions disclosed herein are also substantially free of one or more electrolytes. Viscosity of the cleansing compositions disclosed herein are not dependent upon electrolyte inclusion. Such electrolytes include halides of alkaline metals, alkaline earth metals, ammonium and other metals, such as aluminum and zinc; sulphates and phosphates of alkaline metals, alkaline earth metals, ammonium and other metals such has aluminum and zinc; MEA and DEA salts, and alkaline metal silicates, among others. In one embodiment, the cleansing composition is substantially free of electrolytes including sodium chloride, potassium chloride, sodium sulphate, potassium sulphate, magnesium chloride, magnesium sulphate, magnesium chloride, magnesium sulphate, zinc sulphate, ammonium chloride, and MEA chloride, among others and combinations thereof.

Conventional buffers/pH modifiers may be used. It is desirable to include any pH modifier known in the field acceptable for use in personal care and/or pharmaceutical products. Illustrative but nonlimiting pH modifiers include common additives such as organic acids (e.g., citric acid), inorganic acids (e.g., hydrochloric acid), inorganic bases (e.g., sodium hydroxide and potassium hydroxide), and organic bases (e.g., triethanolamine and aminomethyl propanol). These materials are added at amounts to obtain the desired pH of the cleansing composition ranging from 4.0 to 6.0, or 4.15 to 4.75, or 4.25 to 5.5, or 4.5 to 5.0, where pH is determined using a Thermo Fisher Scientific pH meter.

The viscosity of the cleansing composition is typically from 1,000 to 50,000 cPs, or 2,000 to 30,000 cPs, or 3,000 to 30,000 cPs, or 5,000 to 20,000 cPs, or 7,000 to 15,000 cPs. Viscosity may be measured with art recognized instrumentation such as a Discovery HR-2 Hybrid Rheometer or Brookfield Viscometer, RV5 spindle for 30 seconds at 25 °C and 10 rpm.

In one aspect, cleansing compositions disclosed herein are isotropic. Isotropic, as used herein, refers to the structural packing the surfactants in the cleansing composition, as contrasted with lamellar compositions. In another aspect, cleansing compositions disclosed herein are flowable at a temperature of 25 °C and atmospheric pressure.

Fragrances, fixatives, abrasives and additional skin benefit agents may optionally be included in cleansing compositions disclosed herein. Skin benefit agents suitable for use are meant to include but not be limited to opacifiers, colorants, humectants, emollients, occlusive agents, plant extracts, optical agents, skin lightening agents, anti-inflammatory agents, anti-acne agents, antioxidants, sunscreens, photostabilizers, surfactants, wrinkle reducing agents, coloring agents, desquamation promoters, exfoliating agents, mixtures thereof or the like. Each of these substances may range from 0.05 to 5%, or between 0.1 and 3% by weight.

Conventional humectants, generally of the polyhydric alcohol-type materials, may be used in the disclosed cleansing composition. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. In one embodiment, the cleansing composition comprises glycerin, pentylene glycol, propylene glycol, sorbitol, or a mixture thereof. The amount of humectant employed may range anywhere from 0.1 to 20%, or 0.5 and 15%, or between 1 and 10% by weight of the cleansing composition.

Hydrophobic emollients with weight average particle sizes below either 1000 or 500 microns in diameter are defined herein as “finely dispersed oils” and are preferably used at a minimum of 3, 2, 1 or 0% by weight and a maximum of 20, 30, 40 or 50% by weight.

These hydrophobic emollients include but are not limited to the following:
(a) silicone oils and modifications thereof such as linear and cyclic polydimethylsiloxanes; amino, alkyl, alkylaryl, and aryl silicone oils;
(b) fats and oils including natural fats and oils (triglycerides) such as jojoba, soybean, sunflower, safflower, algal, rice bran, avocado, almond, olive, sesame, persic, castor, coconut, mink oils; cacao fat; beef tallow, lard; hardened oils obtained by hydrogenating the aforementioned oils; and synthetic mono-, di- and triglycerides such as myristic acid glyceride and 2-ethylhexanoic acid glyceride;
(c) waxes such as carnauba, spermaceti, beeswax, lanolin, and derivatives thereof;
(d) hydrophobic plant extracts;
(e) hydrocarbons such as petrolatum, polybutene, liquid paraffins, microcrystalline wax, ceresin, squalene, pristan and mineral oil;
(f) higher alcohols such as lauryl, cetyl, stearyl, oleyl, behenyl, cholesterol and 2-hexydecanol alcohol;
(g) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;
(h) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils;
(i) mixtures of any of the foregoing components, and the like.

Preferred emollients include petrolatum; natural wax; partially or fully hydrogenated triglyceride oils; and mixtures thereof. Preferred triglyceride oils include soybean oil or sunflower oil.

Other additional optional skin benefit agents desirable for use in the disclosed cleansing composition include minerals and skin nutrients such as milk; magnesium, calcium, copper, zinc and other metallic components; beta hydroxy acids, e.g., salicylic acid and derivatives thereof (such as 5-octanoyl salicylic acid, heptyloxy 4 salicylic acid, and 4-methoxy salicylic acid); kojic acid; hydroquinone and arbutin; resveratrol; saccharide isomerate; undecylenoyl phenylalanine; resorcinol derivatives (including thiamidol, 4-ethyl resorcinol, 4-isopropyl resorcinol, 4-hexyl resorcinol, 4-cyclopentyl resorcinol, 4-cyclohexyl resorcinol and acylated forms thereof); ceramides (e.g., Ceramide 1, Ceramide 3, Ceramide 3B and Ceramide 6) and pseudoceramides; allantoin; pyroglutamic acid (PCA) salt derivatives including zinc PCA and sodium PCA; and skin benefit acids and derivatives thereof (10- and/or 12-hydroxystearic acid; petroselinic acid; conjugated linoleic acid; octadecanoic acid; hyaluronic acid and its salt derivatives); retinol and derivatives thereof (retinyl propionate, retinyl palmitate, retinyl acetate), vitamins and derivatives thereof (Vitamin B₂, Vitamin B₃ (niacinamide), picolinamide, Vitamin B₅ (panthenol), Vitamin B₆, Vitamin C, ascorbyl phosphate, ascorbyl glycoside, Vitamin D, Vitamin E, tocopheryl acetate, tocopheryl palmitate, tocopheryl linoleate, folic acid, Vitamin K, and biotin), mixtures thereof and the like. Such skin benefit agents, when used, collectively make up from 0.001 to 12%, or 0.05 to 7% by weight of the cleansing composition. Antibacterial agents like terpineol, thymol, and low molecular weight acids may also be optionally included. Other agents suitable for optional use include zinc pyrithione, octopirox, or mixtures thereof.

Traditional fragrance components like eugenol, coumarin, linalyl acetate, citronellal, iris concentrate, terpinyl acetate, terpineol, thymol, pinenes (e.g., alpha and beta pinene) and citronellol may optionally be added to the cleansing composition as well.

Also optionally suitable for use include materials like chelators (e.g., EDTA), opacifiers (like TiO₂, particle size from 50 to 1200 nm, or 50 to 350 nm), kaolin, bentonite, zinc oxide, iron oxide, mica, C₈-22 fatty acid substituted saccharides, lipoic acid, retinoxytrimethylsilane (available from Clariant Corp. under the Silcare 1M-75 trademark), dehydroepiandrosterone (DHEA) or mixtures thereof.

A wide selection of botanical extracts may optionally be included in cleansing compositions disclosed herein. The extracts may either be soluble in water or oil, carried in a solvent that is hydrophilic or hydrophobic, respectively. In one embodiment, water or ethanol are the extract solvents. Illustrative examples include those extracted from green tea, yarrow, chamomile, licorice, aloe vera, citrus unshui, Ginko biloba, ginseng, marigold, hibiscus, willow bark, alfalfa, algae, grape seed, witch hazel, sage, thyme and rosemary, as well as oils such as those derived from sea buckthorn, moringa, argan, avocado, calendula, algal, and marula. Soy extracts may be used and especially when it is desirable to include retinol. Such extracts, when used, are employed individually or collectively in amounts ranging from 0.001 to 12%, or 0.01 to 7%, or 0.02 to 5%, or 0.03 to 4% by weight of the cleansing composition.

The disclosed cleansing composition may also contain particles that are greater than 50 microns in average diameter that help remove dry skin by functioning as physical exfoliants. Not being bound by theory, the degree of exfoliation depends on the size and morphology of the particles. Large and rough particles are usually very harsh and irritating. Very small particles may not serve as effective exfoliants. Such exfoliants used in the art include natural minerals such as silica, talc, calcite, pumice, tricalcium phosphate; seeds such as rice, apricot seeds, etc.; crushed shells such as almond and walnut shells; oatmeal; polymers such as polyethylene and polypropylene beads, flower petals and leaves; microcrystalline wax beads, synthetic wax beads, jojoba ester beads, and the like. These exfoliants come in a variety of particle sizes and morphology ranging from micron-sized to a few millimeters. They also have a range of hardness.

Cleansing compositions disclosed herein may also include natural and synthetic antioxidants. Illustrative but nonlimiting examples of antioxidants for use in the composition include butylated hydroxytoluene (BHT), dilauryl thiodipropionate, pentaerythrityl tetra-di-t-butyl hydroxyhydrocinnamate, and polyphenols. The total weight percent of antioxidants comprised is from 0.01 to 4%, or from 0.02 to 3%, or from 0.05 to 2% by weight of the composition.

Another optional additive suitable for use includes hemp oil with 2.5 to 25% by weight cannabigerol and/or cannabidiol at from 0.5 to 10 percent by weight. When used, such oil makes up from 0.0001 to 12%, or from 0.01 to 5% by weight of the cleansing composition.

Optionally, one or more coloring agent may also be included in the disclosed cleansing compositions. Coloring agents include dyes or pigments of natural or synthetic origin. A dye colorant selected for use may be organic or inorganic and water-soluble or oil-soluble. Cleansing compositions comprise from 0.01 to 20%, or 0.1 to 15%, or 0.5 to 10% by weight of the coloring agent, when used, relative to the total weight of the cleansing composition.

It is within the scope of the disclosed cleansing composition to optionally include sunscreens and photostabilizers. The sunscreens and photostabilizers that may be used include such materials as octylmethoxycinnamate (OMK), ethylhexyl salicylate, phenylbenzimidazole sulfonic acid (Ensulizole), ethylhexyl p-methoxycinnamate, available as Parsol MCX®, Avobenzene (butyl methoxydibenzoylmethane), available as Parsol 1789® and benzophenone-3, also known as oxybenzone. Inorganic sunscreen actives may be employed such as microfine titanium dioxide, zinc oxide, polyethylene, and various other polymers are also desirable. Other sunscreens desirable for use include p-aminobenzoic acid (PABA), octyldimethyl-PABA, 2-ethoxyethyl p-methoxy cinnamate, benzophenone-1, benzophenone-2, benzophenone-6, benzophenone-8, benzophenone-9, benzophenone-12, homomethyl salicylate, menthyl anthranilate, benzophenone-4, triethanolamine salicylate, terephthalylidene dicamphor sulfonic acid, bisoctriazole, bisethylhexyloxyphenol methoxyphenyl triazine, bisdisulizole disodium, diometriazole trisiloxane, octyltriazone, iscotrizinol, polysilicone-15, isopentenyl-4-methoxycinnamate, mixtures thereof or the like. Also suitable for use is octocrylene. Amounts of the sunscreen or photostabilizing agents when present may generally range from 0.1 to 30%, or from 0.5 to 20%, or from 0.75 to 10% by weight of the cleansing composition.

Conditioning agents like hydroxypropyltrimonium chloride, 5-ureidohydantoin and/or glyoxyldiureide may be used. The components when used make up from 0.5 to 4%, or 0.75 to 4%, or 1 to 3% by weight of the cleansing composition.

Preservatives can be incorporated into the disclosed cleansing compositions as desired to protect against the growth of potentially harmful microorganisms. Cosmetic chemists are familiar with appropriate preservatives and routinely choose them to satisfy preservative tests and product stability tests. Preservative systems should be selected having regard for the use of the composition and possible incompatibilities between the preservatives and other ingredients in the formulation. Exemplary examples of preservatives for compositions of this invention include, without limitations, iodopropynyl butyl carbamate (IPBC), phenoxyethanol, ethylhexylglycerine, 1,2-octanediol, methyl paraben, propyl paraben, imidazolidinyl urea, sodium dehydroacetate, propanediol, alkyl esters of para-hydroxybenzoic acid, sodium benzoate, benzoic acid, hydroxyacetophenone, DMDM hydantoin derivatives, propionate salts, and a variety of quaternary ammonium compounds. Preservatives are employed in amounts ranging from 0.01 to 2% by weight of the composition, including all ranges subsumed therein. In another aspect, no traditional preservative is used in the composition if desired giving the consumer the option to have a preservative-free product. In one aspect, the cleansing composition may be free of preservative. In another aspect, the cleansing composition may comprise of a preservative system that is formaldehyde-free, paraben-free, or both.

In one aspect, the cleansing composition is biodegradable, or at least 97% biodegradable. In another aspect, the cleansing composition is substantially free of silicone, sulfates, or a combination thereof. In still another aspect, the cleansing composition does not contain any (i.e., 0% by weight) silicone, sulfate, or both.

Many types of packaging can be used to store and deliver the present cleansing composition. The selection of packaging is dependent upon the end-use and the viscosity of the composition itself. As an example, leave-on lotions and creams for skin typically employ plastic containers with an opening at a dispense end covered by an appropriate closure. Conventional closures include flip-top hinged lids, screw-caps and non-aerosol pumps. As another example, appropriate packaging to be used for antiperspirants, deodorants and depilatories include a container with a roller-ball applicator on a dispensing end if the composition is fluid and of a thinner viscosity. If the composition is in a stick format, a container with a propel-repel mechanism wherein the stick is fixed on a platform towards a dispensing orifice is appropriate. If the composition is in an aerosol format, then metallic cans pressurized by a propellant and having a spray nozzle is appropriate. In general, patches, bottles, tubes, roller-ball applicators, squeeze containers or lidded jars are preferred.

The term “derivative(s)” as used with reference to ingredients herein refers to compounds which may be formed from a precursor compound and/or is a structural or chemical analog. All states of matter, as used herein, such as solid, liquid and gas relate to the specified state of matter at 25 °C and atmospheric pressure.

The term “skin” as used herein includes the skin on the face, neck, chest, back, arms, axilla, buttocks, hands, legs and scalp. Skin benefit agent, as used herein, is meant to include a component that improves a facial or body characteristic after topical application like a skin characteristic and/or benefits the same wherein the skin benefit agent can be incorporated in a desirable format.

The following Examples are provided to further illustrate an understanding of the invention. The Examples are not intended to limit the scope of the claims. One of ordinary skill in the art will recognize that variations of the method steps that differ from the examples given may be practiced without deviating from the teachings of the present compositions.

### EXAMPLES

All combinations were made by mixing with moderate shear the mentioned active ingredients under conditions of about 25 to 85 °C, and atmospheric pressure.

### Example 1. Sample Formulations

Cleansing compositions were made according to the present invention with the ingredients and amounts set forth in Table I as four different samples. The ingredients were combined via moderate shear/agitation at atmospheric pressure and at temperature of 25 to 85 °C.

TABLE I
Cleansing compositions

| | Weight Percent (wt %) | | | |
|---|---|---|---|---|
| Ingredient Name | A | B | C | D |
| Water | Balance | Balance | Balance | Balance |
| Cocamidopropyl betaine | 22.8 | 22.8 | 22.8 | 22.8 |
| Humectant | 7.00 | 1.50 | 7.00 | 7.00 |
| Lactic Acid | 5.68 | — | 5.68 | 5.68 |
| Sodium methyl lauroyl taurate | 4.00 | 4.00 | 4.00 | 4.00 |
| Preservative | 0.49 | 0.40 | 0.49 | 0.49 |
| Stearic acid | 0.05 | 0.05 | 0.05 | 0.05 |
| pH adjuster | 3.36 | 0.02 | 3.35 | 3.35 |
| Guar hydroxypropyltrimonium | — | 0.35 | 0.35 | 0.35 |
| chloride | | | | |
| Cocamide MEA | — | 0.59 | — | 0.59 |
| Sclerotium gum | — | — | 0.50 | 0.30 |
| Skin benefit agents | 0.02 | 0.02 | 0.2 | 0.02 |
| TOTAL | 100 | 100 | 100 | 100 |
| Stability observations | Stable | Stable | Not stable, guar | Not stable, |
| | | | precipitates out at | sclerotium gum |
| | | | high temperature | precipitated out at 2 |
| | | | conditions, 45/50 °C. | weeks, 37 °C |
| | | | This sample was also | |
| | | | too thick (viscous). | |

Sample A was made according to the disclosed cleansing composition. Surprisingly, Sample A is stable and possesses a desirable viscosity without having electrolyte and thickening agent. Sample B was not made according to the disclosed cleansing composition. Specifically, Sample B does not contain any organic acids but does comprise guar hydroxypropyltrimonium chloride, and so serves as a control with guar. Samples C and D were not made according to the disclosed cleansing composition. On stability, precipitation of guar was observed in Sample C and the resultant composition did not have a desirable viscosity, while precipitation of sclerotium gum was observed in Sample D.

### Example 2. pH Curves

The ingredients of the formulae made in Example 2 were mixed in a manner similar to those described in Example 1, based on Sample A. The level of lactic acid was held constant in Example 2 at an active level of 5%.

TABLE I

| | | | | Temperature |
|---|---|---|---|---|
| | | pH | Viscosity | (°C) |
| | Before lactic acid addition | 5.90 | 0 | 28.0 |
| | After lactic acid addition | 2.80 | 760 | 25.4 |
| NaOH | 0.00 | 2.87 | 1080 | 25.0 |
| wt % | 2.40 | 4.16 | 4560 | 24.2 |
| (50% | 2.40 | 4.33 | 9040 | 20.0 |
| solution) | 2.50 | 4.40 | 10320 | 20.0 |
| | 2.60 | 4.40 | 10560 | 20.3 |
| | 2.70 | 4.48 | 11120 | 20.6 |
| | 2.80 | 4.56 | 12400 | 21.0 |
| | 2.90 | 4.62 | 13600 | 21.0 |
| | 3.00 | 4.68 | 14360 | 21.4 |
| | 3.10 | 4.77 | 14880 | 21.5 |
| | 3.20 | 4.86 | 15560 | 21.6 |
| | 3.30 | 4.95 | 15000 | 22.3 |

TABLE II

| | | | | Temperature |
|---|---|---|---|---|
| | | pH | Viscosity | (°C) |
| | Before lactic acid addition | 6.12 | 0 | 22.7 |
| | After lactic acid addition | 3.05 | 1000 | 23.0 |
| NaOH | 0.00 | 3.01 | 1320 | 21.2 |
| wt % | 0.25 | 3.24 | 1320 | 21.4 |
| (50% | 0.50 | 3.33 | 1160 | 22.0 |
| solution) | 0.75 | 3.47 | 1440 | 22.0 |
| | 1.00 | 3.60 | 1560 | 22.2 |
| | 1.25 | 3.72 | 1840 | 22.2 |
| | 1.50 | 3.77 | 2160 | 22.2 |
| | 1.75 | 3.88 | 2720 | 22.4 |
| | 2.00 | 4.06 | 3920 | 22.9 |
| | 2.25 | 4.20 | 5360 | 23.0 |
| | 2.50 | 4.40 | 7320 | 22.6 |
| | 2.75 | 4.52 | 10120 | 23.0 |
| | 3.00 | 4.78 | 12160 | 22.7 |
| | 3.25 | 4.93 | 13360 | 22.8 |
| | 3.50 | 5.49 | 9400 | 22.7 |
| | 3.75 | 6.10 | 960 | 22.4 |
| | 4.00 | 9.13 | 560 | 22.7 |

The data in Example 2 unexpectedly demonstrates that the viscosity of the disclosed cleansing composition was stable without having thickening agent and/or electrolyte at the noted pH values, wherein desirable viscosities are observed in pH ranging from 4.0 to 6.0, or 4.25 to 5.5. Viscosity was measured using a Brookfield viscometer (i.e., with RV5 spindle at a setting of 10 rpm for 30 s @ 25 °C.

### Example 3. Salt Curve Analysis

The ingredients of the formulae made in Example 3 were mixed in a manner similar to those described in Example 1, based on Sample A. The level of lactic acid was held constant in Example 3 at an active level of 5%. A salt curve was run as per conventional techniques.

TABLE I

| | Viscosity (RV5 spindle, |
|---|---|
| Electrolyte Level (%) | 10 rpm, 30 s @ 25 °C) (cps) |
| 1.03 | 3520 |
| 1.23 | 4080 |
| 1.43 | 4320 |
| 1.63 | 4600 |
| 1.83 | 4640 |
| 2.03 | 4480 |

It was unexpectedly determined that cleansing compositions made according to the compositions disclosed herein in Example 3 illustrates that the inclusion of electrolytes does not serve to thicken the formulation as in other isotropic formulations commercially available. Example 3 employs sodium chloride as the electrolyte analyzed. The pH of the sample evaluated in Example 3 has a pH of 4.45.

Viscosity was measured using a Brookfield viscometer (i.e., with RV5 spindle at a setting of 10 rpm for 30 s @ 25 °C.

1. A cleansing composition comprising:
(a) 0.001 to 20% anionic surfactant by weight of the cleansing composition;
(b) 5 to 40% zwitterionic surfactant by weight of the cleansing composition; and
(c) 1 to 6% alpha hydroxy acid by weight of the cleansing composition;
wherein the cleansing composition has a pH ranging from 4.0 to 6.0,
wherein the cleansing composition is substantially free of electrolytes,
wherein the cleansing composition is substantially free of thickening agent,
and further wherein the anionic surfactant comprises at least a taurate and the zwitterionic surfactant comprises at least a betaine.

2. The cleansing composition according to claim 1, wherein the anionic surfactant comprises isethionate, taurate, sodium alkyl sulfate, acyl glutamate, glycinate, alaninate, or a mixture thereof.

3. The cleansing composition according to claim 1, wherein the composition comprises 0.01 to 15% anionic surfactant by weight of the cleansing composition.

4. The cleansing composition according to claim 1, wherein the anionic surfactant comprises sodium cocoyl alaninate, sodium cocoyl isethionate, sodium lauryl isethionate, sodium methyl cocoyl taurate, sodium methyl lauroyl taurate, sodium lauryl sulfate, sodium lauroyl glutamate, sodium cocoyl glutamate, sodium cocoyl glycinate, potassium cocoyl glycinate, disodium lauryl sulfosuccinate, sodium stearoyl lactylate, or mixtures thereof.

5. The cleansing composition according to claim 1, wherein the zwitterionic surfactant comprises includes-lauryl betaine, betaine citrate, sodium hydroxymethylglycinate, (carboxymethyl)dimethyl-3-[(1-oxododecyl) amino]propylammonium hydroxide, coco alkyldimethyl betaine, cocamidopropyl betaine, (carboxymethyl)dimethyloleylammonium hydroxide, (carboxylatomethyl) dimethyl(octadecyl)ammonium, cocamidopropyl hydroxysultaine, or a mixture thereof.

6. The cleansing composition according to claim 1, wherein the composition comprises 10 to 35% zwitterionic surfactant by weight of the cleansing composition.

7. (canceled)

8. The cleansing composition according to claim 1, wherein the alpha hydroxy acid possesses an acid dissociation constant (pKₐ) from 2 to 12.

9. The cleansing composition according to claim 1, wherein the composition has a viscosity ranging from 1,000 to 50,000 cPs as measured by a viscometer (RV5 spindle, 10 rpm, 30 s @ 25 °C).

10. The cleansing composition according to claim 1, wherein the composition is at least 97% biodegradable, substantially free of silicone, substantially free of sulfates, substantially free of preservatives, or a combination thereof.

11. (canceled)

12. A method of cleansing for gentle exfoliation of the skin comprising the steps of:
(a) contacting the skin with a cleansing composition comprising anionic surfactant, zwitterionic surfactant, and alpha hydroxy acid or derivative thereof, wherein the composition is substantially free of thickening agent, electrolytes, or both; and
(b) removing, or washing or wiping, the cleansing composition off the skin.

13. The cleansing composition according to claim 1, wherein the anionic surfactant consists of taurate and the zwitterionic surfactant consists of betaine.

14. The cleansing composition according to claim 1, wherein the pH ranges from 4.15 to 4.75.

15. The cleansing composition according to claim 1, wherein the pH ranges from 4.25 to 5.5.

16. The cleansing composition according to claim 1, wherein the pH ranges from 4.5 to 5.0.

17. The cleansing composition according to claim 3, comprising 0.5 to 12% anionic surfactant by weight of the cleansing composition.

18. The cleansing composition according to claim 6, comprising 12 to 30% zwitterionic surfactant by weight of the cleansing composition.

19. The cleansing composition according to claim 8, wherein the alpha hydroxy acid possesses an acid dissociation constant (pKₐ) from 2 to 8.

20. The cleansing composition according to claim 16, wherein the alpha hydroxy acid possesses an acid dissociation constant (pKₐ) from 2 to 6.

21. The cleansing composition according to claim 9, wherein the composition has a viscosity ranging from 2,000 to 30,000 cPs as measured by a viscometer (RV5 spindle, 10 rpm, 30 s @ 25 °C).

22. The cleansing composition according to claim 10, wherein the composition is biodegradable.
